(19) United States
(12) Patent Application Publication
(10) Pub. No.: US 2022/0384004 A1
(43) Pub. Date: Dec. 1, 2022

(54) SYSTEM AND METHOD FOR BEHAVIORAL ANOMALY DETECTION BASED ON AN ADHERENCE VOLATILITY METRIC

(71) Applicant: OTSUKA PHARMACEUTICAL CO., LTD., Tokyo (JP)

(72) Inventors: Jonathan Roland Knights, Rockville, MD (US); Zahra Heidary, Rockville, MD (US)

(21) Appl. No.: 17/621,598

(22) PCT Filed: Jul. 1, 2020
(86) PCT No.: PCT/JP2020/026617

§ 371 Date: Dec. 21, 2021

Related U.S. Application Data
(60) Provisional application No. 62/970,095, filed on Feb. 4, 2020.
(60) Provisional application No. 62/869,525, filed on Jul. 1, 2019.

(51) Int. Cl. G16H 20/10

(52) U.S. Cl. CPC G16H 20/10

(57) ABSTRACT

Methods, systems, apparatus, and computer programs, detecting behavioral anomalies in treatment adherence patterns. A method includes actions of obtaining data that represents whether an entity has complied with a therapeutic regimen or has not complied with a therapeutic regimen, determining a central tendency of an adherence volatility metric for the entity for at least n-time periods into the future, determining a plurality of boundaries around the central tendency, determining based on the data represented by the one or more data structures, an current observed adherence volatility metric, determining whether the current observed adherence volatility metric satisfies at least one of the plurality of boundaries around the central tendency, and based on a determination that the current observed adherence volatility metric satisfies at least one of the plurality of boundaries around the central tendency, generating a candidate anomaly data log record, the candidate anomaly data log record including data indicating that a candidate anomaly has been detected.

### CROSS-REFERENCE TO RELATED APPLICATIONS

This application claims the benefit of U.S. Provisional Patent Application No. 62/869,525 filed Jul. 1, 2019. This application also claims the benefit of U.S. Provisional Patent Application No. 62,970,095 filed Feb. 4, 2020. The entire contents of each of these applications is hereby incorporated by reference in their entireties.

### BACKGROUND ART

Digital medicine relates to the marriage between active pharmaceuticals and wearable/ingestible sensors combined with mobile and web-based tools in the hope of improving the management of medication adherence.

### SUMMARY OF INVENTION

According to one innovative aspect of the present disclosure, a method for detecting behavioral anomalies in treatment adherence patterns is disclosed. In one aspect, a method includes obtaining, by one or more computers, one or more first data structures having fields structuring data that represents (i) an indication that an entity has complied with a therapeutic regimen or (ii) an indication that the entity has not complied with the therapeutic regimen, determining, by the one or more computers, an initial volatility metric based on the data represented by the one or more first data structures, determining, by the one or more computers, a central tendency of the initial adherence volatility metric for the entity for at least n-time periods into the future, where n is any non-zero integer, determining, by the one or more computers, a plurality of boundaries around the central tendency, the plurality of boundaries including a first threshold representing an upper bound of the central tendency and a second threshold representing a lower bound of the central tendency, obtaining, by the one or more computers, one or more second data structures having fields structuring data that represents (i) a subsequent indication that an entity has complied with a therapeutic regimen or (ii) a subsequent indication that the entity has not complied with the therapeutic regimen, determining, by the one or more computers and based on the data represented by the one or more second data structures, a current observed adherence volatility metric, determining, by the one or more computers, whether the current observed volatility metric satisfies the first threshold or the second threshold, and based on a determination, by the one or more computers, that the current volatility metric satisfies the first threshold or the second threshold, generating a candidate anomaly data log record, the candidate anomaly data log record including data indicating that a candidate anomaly has been detected.

Other versions include corresponding systems, apparatuses, and computer programs to perform the actions of methods defined by instructions encoded on computer readable storage devices.

These and other versions may optionally include one or more of the following features. For instance, in some implementations the data that represents (i) an indication that an entity has complied with a therapeutic regimen or (ii) an indication that the entity has not complied with the therapeutic regimen can include data that represents (a) an occurrence of an ingestion of a substance by the entity or (b) an absence of ingestion of a substance by the entity, and the data that represents (i) a subsequent indication that an entity has complied with a therapeutic regimen or (ii) a subsequent indication that the entity has not complied with the therapeutic regimen can include data that represents (a) a subsequent occurrence of an ingestion of a substance by the entity or (b) a subsequent absence of ingestion of a substance by the entity.

In some implementations, the one or more first data structures or one or more second data structures were generated, and transmitted, by a mobile device based on ingestion data generated by a patch coupled to the entity.

In some implementations, the patch generated the ingestion data based on detection, by the patch, of a signal from an ingestible sensor in the substance.

In some implementations, the substance can include a medicine.

In some implementations, the upper bound and the lower bound define a region of acceptable adherence volatility metrics.

In some implementations, determining, by the one or more computers, whether the current observed volatility metric satisfies the first threshold or the second threshold can include continuously obtaining data representing an observed volatility metric, and comparing the continuously obtained data to the boundaries defined by the first threshold and the second threshold to determine whether the continuously obtained data falls within the region of acceptable adherence volatility metrics.

In some implementations, determining, by the one or more computers, whether the current observed volatility metric satisfies the first threshold or the second threshold can include evaluating the current observed volatility metric using a binary Markov Chain model to determine whether the current observed volatility metric has exceed the first threshold or the second threshold.

In some implementations, the adherence volatility metric is based on an entropy rate of Markov parameters.

In some implementations, the n-time periods into the future includes n-days into the future.

In some implementations, the n-time periods into the future includes n-hours into the future.

These, and other innovative aspects the present disclosure, are described in more detail in the written description, the drawings, and the claims.

### BRIEF DESCRIPTION OF DRAWINGS

FIG. 1 is a contextual diagram of a system for detecting behavioral anomalies using an adherence volatility metric.

FIG. 2 is a flowchart of a process for detecting behavioral anomalies using an adherence volatility metric.

FIG. 3 is a block diagram of system components that can be used to implement a system for detecting behavioral anomalies using an adherence volatility metric.

### DESCRIPTION OF EMBODIMENTS

The present disclosure is directed towards methods, systems, apparatuses, and computer programs for detecting behavioral anomalies in treatment adherence patterns. In some aspects, the present disclosure can be leveraged in real-time for highlighting relative behavioral anomalies at the individual entity level. A behavior anomaly, or anomaly, in accordance with the present disclosure means a change or shift in individual entity behavior related to a treatment plan. A treatment plan can include, for example, a medication regimen. However, though one practical application of the disclosed anomaly detection method can include detecting anomalies in historically observed patient data, the present disclosure should not be so limited. Instead, the disclosed anomaly detection method can be applied to any binary data series having properties fitting of a Markov model.

Advantages of the present disclosure include an anomaly detection system and method that does not require prior training of a model. Instead, a patient's own evolving behavior, referred to herein as adherence volatility and represented, for example, by an adherence volatility metric trace, is used to construct expectation bounds at multiple future intervals. These constructed expectation bounds can then be monitored with respect to a currently observed volatility metric for an entity to detect anomalies without need for training or relying on a difference from any reference sequence.

Another advantage of the present disclosure over conventional systems is that future intervals that define the expectation bounds can be dynamically updated using newly received and analyzed observation data such as ingestion data. Thus, the system of the present disclosure can generate new future intervals defining the expectation bounds as new data is received, thereby allowing the expectation bounds to evolve over time based on newly received data. In some implementations, the future intervals that define the expectation bounds can be determined using binary Markov chains.

However, the present disclosure is not limited to two states determined using binary Markov chains. Instead, in some implementations, data having three or more states can be monitored and a multi-state Markov chain used to determine evolving future values for the respective states, for example, if the process is irreducible and homogenous>.

The process for anomaly detection can begin by using one or more computers to obtain one or more data structures having fields structuring data that represents whether an entity has complied with a therapeutic regimen or not complied with a therapeutic regimen. In some implementations, such data can include data representing (i) an occurrence or (ii) an absence of ingestion of a substance by an entity. The one or more computers can include one or more cloud-based, or otherwise networked, computers. The one or more computers can be configured to obtain the one or more data structures from one or more mobile devices such as a smartphone, tablet, smartwatch, or the like associated with an entity. The mobile device can be configured to generate the one or more data structures structuring data representing the occurrence or absence of ingestion of a substance based on ingestion data generated by a patch coupled to the entity. The patch can be configured to generate the ingestion data based on detection, by the patch, of a signal from an ingestible sensor in the substance. The substance can include a medicine.

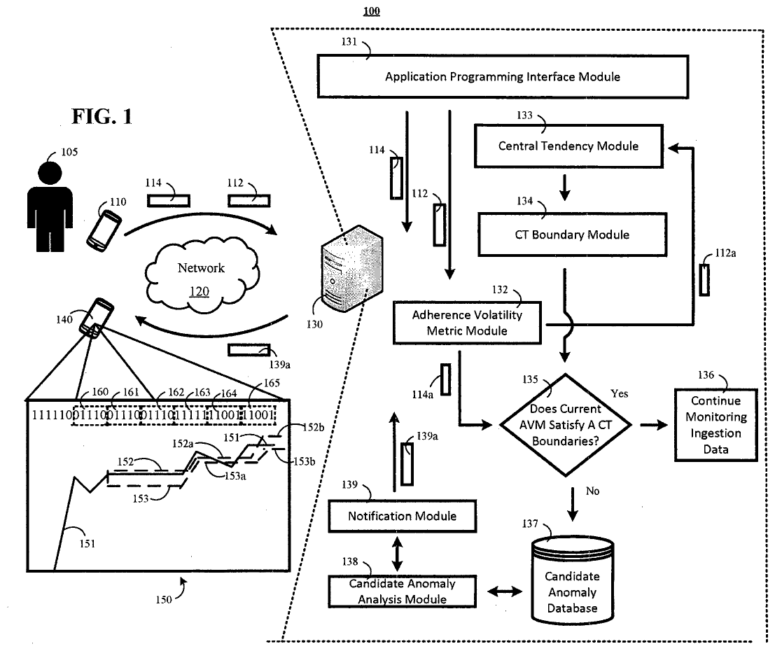

FIG. 1 is a contextual diagram of a system 100 for detecting behavioral anomalies using an adherence volatility metric. The system 100 can include a first user device 110, a network 120, an application server 130, and a second user device 140.

In the example of FIG. 1, an entity such as a person 105 has begun a regimen such as a medicinal regimen. For example, the person 105 can begin taking a prescribed medicine. A first user device 110 can be used to collect observation data 112, 114 describing the person's 105 participation in the regimen and transmit the collected observation data 112, 114 describing the person's 105 participation in the regimen to the application server 130 via the network 120. The network 120 can include a wired Ethernet network, an optical network, a WiFi network, a LAN, a WAN, a cellular network, the Internet, or any combination thereof.

The first user device 110 is depicted as a smartphone for sake of the illustration. And, in some implementations, the first user device 110 can be a smartphone. For example, a smartphone can collect data describing the person's 105 participation in a regimen in a number of ways such as by syncing with one or more wearable devices that broadcast data describing the person's 105 participation in the regimen using shortwave radio signals such as Bluetooth. Then, the smartphone can transmit the observation data 112, 114 describing the person's 105 participation in the regimen to the application server 130. However, the present disclosure is not limited to a user device 110 that is a smartphone.

For example, in some implementations, the user device 110 can be any wearable device such as smartwatch, a patch that adheres to the person's 105 skin, a form of clothing having internet of things (JOT) sensors, or the like. In such implementations, the user device 110 can be capable of obtaining data describing the person's 105 participation in the regimen and transmitting the data describing the person's 105 participation in the regimen to the application server 130 without first transmitting the data describing the person's 105 participation in the regimen to another user device.

The application server 130 can include a plurality processing modules. For example, the application server 130 can include an application programming interface (“API”) module 131, an adherence volatility module 132, a central tendency module 133, a CT Boundary Module 134, a decisioning module 135, a candidate anomaly analysis module 138, and a notification module 139. In addition, the application server 130 can include, or otherwise have access to, a candidate anomaly database 137. For purposes of this specification, the term module can include one or more software components, one or more hardware components, or any combination thereof, that can be used to realize the functionality attributed to a respective module by this specification.

A software component can include, for example, one or more software instructions that, when executed, cause a computer to realize the functionality attributed to a respective module by this specification. A hardware component can include, for example, one or more processors such as a central processing unit (CPU) or graphical processing unit (GPU) that is configured to execute the software instructions to cause the one or more processors to realize the functionality attributed to a module by this specification, a memory device configured to store the software instructions, or a combination thereof. Alternatively, a hardware component can include one or more circuits such as a field programmable gate array (FPGA), an application specific integrated circuit (ASIC), or the like, that has been configured to perform operations using hardwired logic to realize the functionality attributed to a module by this specification.

With reference to the example of FIG. 1, the system 100 can begin a process of detecting behavioral anomalies using an adherence volatility metric by the application server 130 receiving observation data 112, 114. The observation data 112, 114 can include, for example, data that represents whether the person 105 has complied with a therapeutic regimen or not complied with a therapeutic regimen. In some implementations, a therapeutic regimen can include consumption of a substance such as a medicine by the person 105. In such implementation, the data representing whether the person 105 has complied with the therapeutic regimen can include data representing (i) an occurrence of an ingestion of a substance or (ii) an absence of an ingestion of a substance.

Data describing the occurrence of an ingesting of a substance can include, for example, data generated by a patch that has been coupled to the skin of the person 105 indicating that the person 105 has ingested a substance. The patch can generate this data in response to detection, by the patch, of a data output by a sensor in the stomach of a person which has been embedded into a medicine that was ingested by the person. The data generated by the patch can be data 112, 114 and can be transmitted by the patch to the application server 130 using the network. In such an implementation, patch can be the user device 110. In other implementations, the data generated by the patch can be detected by a user device 110 such as a smartphone or smartwatch, and then the user device 110 can transmit the detected observation data 112, 114 to the application server 130.

Data indicating the occurrence of an ingestion of a substance can be observation data such as observation 112 or 114. Data describing the occurrence of an absence of an ingestion of a substance can be generated by the patch, the user device 110, or both, indicating that the patch, the user device 110, or both, has not detected data indicating the occurrence of an ingestion of a substance for more than a threshold amount of time. For example, if no ingestion is detected for a 24 hour time period, then the patch, the user device 110, or both, can generate data indicating the absence of an ingestion of a substance. Data indicating the absence of an ingestion of a substance can be observation data such as observation data 112 or 114.

However, the present disclosure need not be so limited. Instead, in some implementations, the observation data 112, 114, provided to the application server 130 can indicate whether or not data representing (i) an occurrence of an ingestion of a substance or (ii) an absence of an ingestion of a substance has been obtained. In some implementations, the therapeutic regimen can include consumption of multiple substances by a person, consumption of a substance and performance of physical or mental exercises, or merely just performance of physical or mental exercises. In each implementation, observation data 112, 114 can be generated that indicates whether the person 105 complied with the therapeutic regimen or did not comply with the therapeutic regimen.

In some implementations, such as with the therapeutic regimen of five medications that the person 105 must ingest, the system 100 can generate data indicating whether the person 105 complied with the therapeutic regimen or did not comply with the therapeutic regimen in a number of different ways. For example, in one particular implementation, the system 100 may generate data indicating that the person 105 complied with the therapeutic regimen if data was obtained indicating that the person 105 ingested all five of the medicines in a particular time period. However, in another implementation, the system 100 can generate data indicating that the person complied with the therapeutic regimen if the person 105 ingested more than a threshold amount of the 5 medicines. Multiple other implementations may also fall within the scope of the present disclosure.

Continuing with the example of FIG. 1, the application server 130 can receive the observation data 112, 114 using an application programming interface module (API) 131. The API 131 can include software, hardware, or a combination thereof that functions as interface between the user device 110 or user device 140 and the application server 130. For example, the API can receive observation data such as observation data 112, 114 from different user devices such as user devices 110 of respective different entities. In addition, the API 131 can function to provide notifications to the user device 110 or to another user device 140 after using the processing modules of the application server 130 to execute a process such as the process 200. The application server 130 can process observation data 112, calculate adherence volatility metrics 112a, 114a based on the observation data 112, 113, determine a central tendency of the calculated adherence volatility metric 112a, determine a plurality of boundaries around the central tendency, and then determine whether a candidate behavioral anomaly occurred based on whether a current adherence volatility metric such as current volatility metric 114a satisfies at least one of the plurality of boundaries.

With reference to the example of FIG. 1, the application server can receive observation data 112 using the API 131. The observation data 112 can include observation data indicating that an ingestion was observed or not observed for a single time period such as during a one hour time period, a four hour time period, a twenty-four hour time period, or the like. Alternatively, the observation data 112 can include observation data indicating that an ingestion was observed or not observed for a multiple sequential time periods such as 5 one hour time periods, 5 four hour time periods, 5 twenty-four hour time periods, or the like. The API 131 can provide the observation data 112 to the adherence volatility metric module 132. The adherence volatility metric module 132 can calculate an adherence volatility for a person 105 based on observation data such as observation data 112. Adherence volatility, which may be represented as a numerical value referred to herein as an adherence volatility metric, is a numerical value that represents a degree to which substance ingestion behavior fits expected behavior based on historically observed data.

In some implementations, the adherence volatility module 132 can generate a representation of adherence volatility, referred to as an adherence volatility metric, by determining a longitudinal evolution of the entropy rate of a single binary Markov chain generated from observation data generated during a person's treatment with a particular medicine. In this example, observation data can include a success state such as “1” indicating an observed ingestion on a given day or an unobserved state such as “0” indicating that an ingestion on a given day was unsuccessful or not observed. Use of an entropy rate to represent adherence volatility can provide information as to shifts in both the marginal (stationary) and conditional dependence structures simultaneously, making it a promising measure by which to detect behavioral (contextual) anomalies.

In some implementations, a binary Markov Chain can be used to determine an entropy rate representation of adherence volatility. For a binary Markov Chain (assumed to be stationary and irreducible), the entropy rate is defined as:

$$H ( X ) = - {\sum }_{q , r ∈ \{ 0 , 1 \}} {π}_{q} * {p}_{q , r} log ( {p}_{q , r} )$$

where π_{q} is the stationary distribution of each state q∈{0,1} representing

$${lim}_{T \to ∞} P ( {X}_{T - 1} = q ) .$$

The logarithm term in this implementation refers to the natural logarithm. For a subject i on day T, the observed Markov chain is represented as [symbol] ᵢ^{T}=[x₁, x₂, . . . , x_{T}] where xₜ∈{0,1} represents whether or not an ingestion was observed (1) or not (0) on day t. The two-state Markov chain for this subject—up to day T— can be represented by the transition matrix:

$${A}_{i}^{T} = [ \begin{matrix}{p}_{i , 00}^{T} & {p}_{i , 01}^{T} \\ {p}_{i , 10}^{T} & {p}_{i , 11}^{T}\end{matrix} ]$$

capturing the observed probabilities of ingestion successes and failures to be followed by success or failure. In some implementations, the transition probabilities are represented using the maximum-likelihood definition of p_{q,r}=n_{q,r}/n_{q+}. An estimate for the entropy rate of this Markov chain under these conditions is then:

$$= H ( {A}_{i}^{T} ) = - {\sum }_{q , r ∈ \{ 0 , 1 \}} {π}_{i , q}^{T} ( {p}_{i , qr}^{T} log {p}_{i , qr}^{T} )$$

In some implementations, the stationary distribution π_{i,q}^{T} can be estimated using the eigenvalue decomposition method of [symbol] . In such implementations, adherence volatility for subject i is represented as the longitudinal evolution of [symbol] .

The application server 130 can provide the adherence volatility metric 112a generated by the adherence volatility metric module 132 as an input to the central tendency module 133. The central tendency module 133 is configured to take the input of an adherence volatility metric 112a and determine a central tendency of the adherence volatility metric 112a for the person 105 for at least n-time periods into the future, where n is any non-zero integer. An n-time period can include n-hours, n-days, n-weeks, or the like into the future, where n is any non-zero integer. The central tendency thus serves as an estimation of a set of observation data n-time periods into the future. For example, in some implementations, the central tendency of the adherence volatility metric, which in some implementations can be represented as entropy rate of observation data, can be calculated as a weighted average of all possible entropy rates for the person 105 n-days into the future. This central tendency is thus an estimated future adherence of a person 105 to a regimen such a medicinal regimen that includes ingestion of a substance for n-days into the future given an existing measure adherence volatility for the person 105 that is based on historical observation data describing the person's 105 ingestion behavior.

The central tendency (CT) boundary module 134 is configured to determine a plurality of boundary thresholds around the central tendency of adherence volatility determined by the central tendency module 132. The plurality of boundary thresholds can include a first boundary threshold that is greater than the estimated central tendency and a second boundary threshold that is less than the estimated central tendency threshold. The boundary thresholds are dynamically calculated, on a future interval basis, based on variations in the person's 105 historical adherence evidenced by the adherence volatility metric 112a used to calculate the central tendency.

In some implementations, each of the future intervals may correspond to set number of time periods such as 5 one hour time periods, 5 four hour time periods, 5 twenty-four hour time periods, or the like and can correspond to the value n. The boundaries define an expected level of entropy rate variation from the central tendency for a further interval of n time periods. A decisioning modules 135 can determine whether subsequent entropy rates representing an adherence volatility of the person 105 for a particular interval of time satisfies the bounds for the particular interval. If subsequent entropy rates determined based on observation from a user device satisfy one of these bounds, a log record can created that indicates the detection of candidate behavioral anomaly and stored in a candidate anomaly database 137. A behavioral anomaly can be include a shift in the persons' 105 adherence to a medicinal regimen. Importantly, these bounds can be dynamically recalculated and updated at respective intervals of n. This enables the system 100 to dynamically adapt to behavioral ingestion patterns that are normal to the person 105 without being trained in advance.

Here, a static time period of future intervals is described as being of duration n. in this implementations, each of the future time intervals are the same duration n. However, the present disclosure need not be so limited. For example, in some implementations there is no requirement that future intervals be limited to time periods of static duration. Instead, in some implementations, future intervals can be used that are each of different lengths. For example, a first future interval may be a three day time period, second future interval may be a 6 day time period, a third future interval may be a 2 day time period, and the like.

This use of dynamically adapting boundary criteria enables a system for contextual anomaly detection that can be used, in some implementations, for adaptive outlier detection. A pseudocode algorithm for this boundary determination process is set forth below in Table 1.

TABLE 1
VARIABLES

[symbol]

^{t} ← observed data of length t
c ← Initial observation duration
n ← anomaly observation window length
S = {sᵢ}:i ≤ 2ⁿ → Set of 2ⁿ possible futures [symbol]
ₛ_{ᵢ}^{t} ← concatenation of [symbol] ^{t} and sᵢ
Wⁱ = {wᵢ^{t}} where wᵢ^{t} = P(sᵢ| [symbol] ^{t})
(NB: It follows directly that Σ_{w}_{^{t}} = 1)

FUNCTIONS

```
def window_bounds( [symbol] ^{D}, m):
  calculate W^{D}
  win_{avg} = Σᵢ₌₁²ⁿ wᵢ^{D} * Ĥ( [symbol] ₛ_{ᵢ}^{D})
  V2 = Σᵢ₌₁²ⁿ (wᵢ^{D})²
  ${win}_{sd} = \sqrt{\frac{1}{1 - V 2} * {\sum }_{i = 1}^{{2}^{n}} {w}_{i}^{D} * {( \hat{H} ( {x}_{{s}_{i}}^{D} ) - {win}_{avg} )}^{2}}$
  return {win_{avg} − m * win_{sd} )_{,} (win_{avg} + m * win_{sd})
```

PSEUDOCODE

```
initialize [symbol]
observe data to obtain [symbol] ^{D} = { [symbol] ᵢ^{D}} : D > c
win_bounds = dict( )
win_num = [symbol]
for i in c:D:
      calculate h = Ĥ( [symbol] ⁱ)
  if (i − c) mod n = = [symbol] :
    # border point: window bounds for next window
    win_num += 1
    win_min, win_max = window_bounds( [symbol] ⁱ, 1)
    win_bounds (win_num] = [win_min, win_max]
    if (h > win_bounds[win_num−1]) or /
          (h < win_bounds [win_num−1]):
      register anomaly
else:
  if (h > win_bounds[win_num]) or /
            (h < win_bounds [win_num]):
      register anomaly
```

In more detail, after an initial observation period, the central tendency of the adherence entropy rate observations for the next ‘n’ time periods such as n-days is calculated as a weighted average of all possible entropy rates n days into the future. In some implementations, the initial observation period may be a predetermined amount of time such as 24 hours/one day. However, the present disclosure need not be limited to such a time period for an initial observation and in some implementation the initial observation period can be less time or more time than 24 hours/one day. For a binary Markov Chain and an n-day observation window, there are 2ⁿ possible future states. The weights are calculated as the probability of each event given the historically observed data to that point. In some implementations, expectation on the boundaries around the central tendency can be set to 1 standard deviation calculated from the observed weighted variance. Accordingly, the present disclosure can be used to generate boundaries for the expected central tendency and variation in the observed entropy rate over the next ‘n’ days, simultaneously.

Once expectation boundaries have been set, or during the calculation of these expectation boundaries, for a particular observation window of the next n-time periods, the application server 130 can continue to observe observation data for the next n-time periods. This can include receiving current observation data such as current observation data 114. The current observation data 114 is observation data that is generated based on ingestion observations that occur at a point in time that is after the ingestion observations on which observation data 112 is based. The API 131 can receive the current observation data 114 and use the adherence volatility metric module 132 to determine a current adherence volatility metric 114a. The current adherence volatility metric 114a can be determined by calculating an entropy rate of the observation data 114. In some implementations, the entropy rate may be determined using a binary Markov chain.

The application server 130 can use decisioning logic 135 to determine whether the current adherence volatility metric 132 satisfies one or more of the plurality of boundaries around the central tendency defining an expected adherence volatility metric variation. If it is determined, by the decision logic, that the current adherence volatility metric 133 does not satisfy one or more of the plurality of boundaries, then the application server 130 can execute programmed logic of module 136 that continues to monitor observation data describing ingestions of the person 105. This can include, for example, obtaining a subsequent set of observation data, generating a subsequent adherence volatility metric, and testing the subsequent adherence volatility metric at the decisioning logic 135. This cycle can continue until the n-time period window expires. At the expiration of the n-time period window, a subsequent n-time period window can be determined, subsequent observation data can be obtained, and the process can continue to iterate, as described above.

Alternatively, if the application server 130 determines, using decisioning logic 135, that the current adherence volatility metric 133 does satisfy one or more of the plurality of boundaries, the then the application server 130 can store a candidate anomaly log record in the candidate anomaly database 137. The candidate anomaly log record can include any data describing the person's state at or near the time when the candidate anomaly log record is created. For example, the candidate anomaly log record can include data describing the observation(s) data 114 on which the current adherence volatility metric is based, the adherence volatility metric, historical observation data from one or more preceding n-time periods, the magnitude of the current boundaries, the like, or any combination thereof. After detection of an candidate anomaly, the application server can execute program logic of module 136 to continue monitoring observation data describing ingestions of the person 105.

In some implementations, the iterative process described above can continue until a terminating criterion is reached. In some implementations, the terminating criterion can be completion of a treatment such as a medicinal regimen. In some implementations, a terminating criterion may include termination of a subscription to a service that can detect behavioral anomalies, as described herein.

The detection of candidate behavioral anomalies, alone, provide significant advantages in the art. This is because it enables user that monitors the person's 105 ingestion behavior to identify potential points in time where the person 105 may begin to deviate from their typical ingestion patterns. The systems and methods described herein are particular innovative over conventional methods in that the boundaries of the central tendency are dynamically determined either after an initial observation period or two after processing one or more observation cycles in a manner that allows dynamic customization of the boundaries to the person's 105 unique behavioral patterns. The dynamic customization occurs as a result of the updating of the central tendency based on the prior window of observations for the user and then updating the boundaries around that central tendency as described herein. Accordingly, systems and methods of the present disclosure are effective and accurate at identifying candidate anomalies that conventional methods.

However, the present disclosure also provides data analysis, notification, and reporting functionality based on the identified candidate anomaly log records stored in the candidate anomaly database 137. For example, in some implementations, the candidate anomaly analysis module 138 can detect newly added candidate anomaly log records stored in the candidate anomaly database 137 and instruct the notification module 139 to generate a notification 139a that that can be transmitted to a user device 110 or 140 using the network 120 to alert a user to the detection of anomaly. In some implementations, the alert can notify a user device 110 of a user. This can include, for example, a pop-up notification that alerts the user that his ingestion pattern may have changed. Such changes may be increases in dosages or missed dosages. Alternatively, the notification 139a can be transmitted to a different user device 140 that may be belong to a physician, nurse, pharmacist, other healthcare professional, or any other user associated with a person's 105 account or profile such as, for example, the person's wife or husband. In some implementations, the notification 139a can be transmitted to a user device 140 for use in downstream predictive modeling.

In some implementations, the candidate anomaly analysis module 138 can also be configured to perform other operations on the candidate anomaly log records. For example, in some implementations, the candidate anomaly analysis module 138 can obtain candidate anomaly log records from the candidate anomaly database 137 and other data collected by the application server 130 or generated by the application server 130. This data can include, for example, historical observation data, central tendency data, boundary data, observation window length data, or the like. The candidate anomaly analysis module 138, or other module of the application server 130 can generate rendering data that, when received and processed by a user device 110, 140 can cause the user device to generate visualizations such as visualization 150. In some implementations, the candidate anomaly analysis module 138 can use the notification module or API to communicate the rendering data to another computer such as user device 150.

The visualization 150 can provide a visual representation of the data analyzed by the application server 130. For example, the visualization 150 can display the central tendency 151 calculated for the person 105, the boundaries 152/153, 152a/153a, 152b/153b, observation data such as a string of 1s and 0s where a “1” represents an ingestion and a “0” represents a non-observed ingestion displayed across the top of the visualization 150, and access windows of n=5 days. In this example, a static time period of n=5 days was used and each observation windows were of the same length. However, the present disclosure need not be so limited. For example, in some implementations there is no requirement that access windows be limited to the time periods or time periods of static duration. Instead, in some implementations, access windows can be used that are each of different lengths. For example, a first time window may be a three day time period, second time window may be a 6 day time period, a third time window may be a 2 day time period, and the like.

Visualization 150 is not shown to scale or mathematically calculated. Instead, it is intended to illustrate concepts related to the present disclosure such as a relatively steady central tendency being maintained, after an initial observation period, as flat and within boundaries 152 and 153 as the user continues with his personal behavioral pattern of “01110” 160, 161, 162 (e.g., day one ingestion not observed, days 2, 3 and 4 ingestion observed, and day 5 non-observed). Then, the behavior at 163 changes and the central tendency adjusts (e.g., upwards), moving it outside the boundaries 152, 153. Then, the boundaries 152, 153 in the next time window can be recalculated to set a new set of boundaries 152a, 1523 around the central tendency.

In yet other implementations, the candidate anomaly analysis module 138 can analyze candidate anomaly log records stored in the candidate anomaly database 137 and determine whether or not a candidate anomaly is an actual anomaly. If the candidate anomaly is determined to be an anomaly, one or more operations can be initiated by the application server 130. For example, the application server 130 can notify the user device 110 or 140 that an actual anomaly has been detected. Alternatively, if the candidate anomaly is not determined to be an anomaly, the application server 130 can determine to not notify the user device 110 or 140 as to the detected candidate anomaly. Such features can significantly reduce bandwidth used to communicate with user devices as well as reduce false notification to user devices 110 or 140.

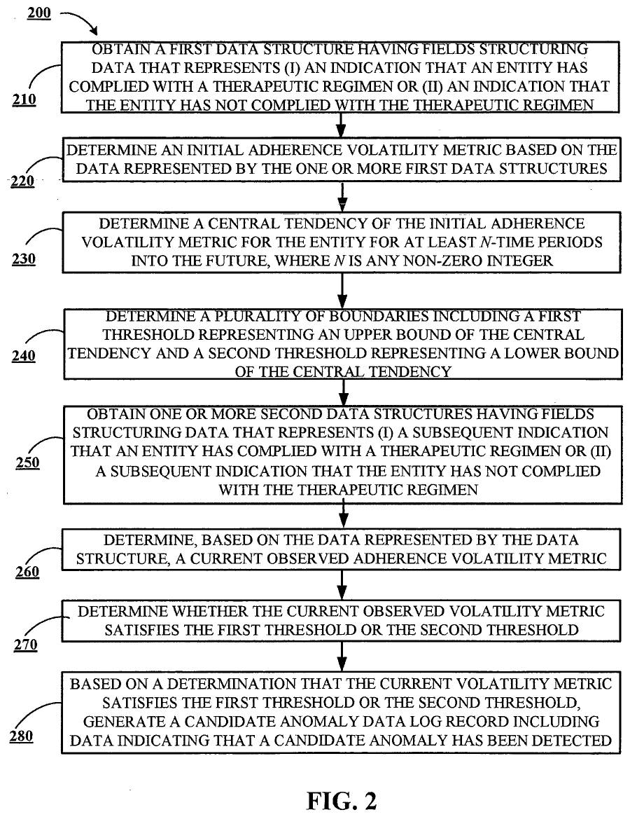

FIG. 2 is a flowchart of a process 200 for detecting behavioral anomalies using an adherence volatility metric. In general, the process 200 can include obtaining, by one or more computers, one or more first data structures having fields structuring data that represents (i) an indication that an entity has complied with a therapeutic regimen or (ii) an indication that the entity has not complied with the therapeutic regimen (210), determining, by the one or more computers, an initial volatility metric based on the data represented by the one or more first data structures (220), determining, by the one or more computers, a central tendency of the initial adherence volatility metric for the entity for at least n-time periods into the future, where n is any non-zero integer (230), determining, by the one or more computers, a plurality of boundaries around the central tendency, the plurality of boundaries including a first threshold representing an upper bound of the central tendency and a second threshold representing a lower bound of the central tendency (240), obtaining, by the one or more computers, one or more second data structures having fields structuring data that represents (i) a subsequent indication that an entity has complied with a therapeutic regimen or (ii) a subsequent indication that the entity has not complied with the therapeutic regimen (250), determining, by the one or more computers and based on the data represented by the one or more second data structures, a current observed adherence volatility metric (260), determining, by the one or more computers, whether the current observed volatility metric satisfies the first threshold or the second threshold (270), and based on a determination, by the one or more computers, that the current volatility metric satisfies the first threshold or the second threshold, generating a candidate anomaly data log record, the candidate anomaly data log record including data indicating that a candidate anomaly has been detected (280).

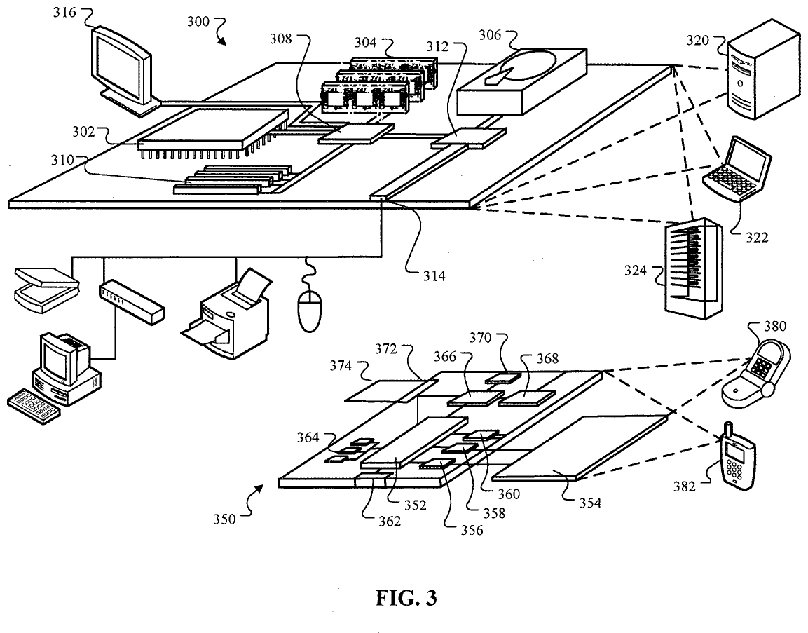

FIG. 3 is a block diagram of system components that can be used to implement a system for detecting behavioral anomalies using an adherence volatility metric.

Computing device 300 is intended to represent various forms of digital computers, such as laptops, desktops, workstations, personal digital assistants, servers, blade servers, mainframes, and other appropriate computers. Computing device 350 is intended to represent various forms of mobile devices, such as personal digital assistants, cellular telephones, smartphones, and other similar computing devices. Additionally, computing device 300 or 350 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 300 includes a processor 302, memory 304, a storage device 306, a high-speed interface 308 connecting to memory 304 and high-speed expansion ports 310, and a low speed interface 312 connecting to low speed bus 314 and storage device 306. Each of the components 302, 304, 306, 308, 310, and 312, are interconnected using various busses, and can be mounted on a common motherboard or in other manners as appropriate. The processor 302 can process instructions for execution within the computing device 300, including instructions stored in the memory 304 or on the storage device 306 to display graphical information for a GUI on an external input/output device, such as display 316 coupled to high speed interface 308. In other implementations, multiple processors and/or multiple buses can be used, as appropriate, along with multiple memories and types of memory. Also, multiple computing devices 300 can be connected, with each device providing portions of the necessary operations, e.g., as a server bank, a group of blade servers, or a multi-processor system.

The memory 304 stores information within the computing device 300. In one implementation, the memory 304 is a volatile memory unit or units. In another implementation, the memory 304 is a non-volatile memory unit or units. The memory 304 can also be another form of computer-readable medium, such as a magnetic or optical disk.

The storage device 306 is capable of providing mass storage for the computing device 300. In one implementation, the storage device 306 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 304, the storage device 306, or memory on processor 302.

The high speed controller 308 manages bandwidth-intensive operations for the computing device 300, while the low speed controller 312 manages lower bandwidth intensive operations. Such allocation of functions is exemplary only. In one implementation, the high-speed controller 308 is coupled to memory 304, display 316, e.g., through a graphics processor or accelerator, and to high-speed expansion ports 310, which can accept various expansion cards (not shown). In the implementation, low-speed controller 312 is coupled to storage device 306 and low-speed expansion port 314. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

Computing device 350 includes a processor 352, memory 364, and an input/output device such as a display 354, a communication interface 366, and a transceiver 368, among other components. The device 350 can also be provided with a storage device, such as a micro-drive or other device, to provide additional storage. Each of the components 350, 352, 364, 354, 366, and 368, are interconnected using various buses, and several of the components can be mounted on a common motherboard or in other manners as appropriate.

The processor 352 can execute instructions within the computing device 350, including instructions stored in the memory 364. The processor can be implemented as a chipset of chips that include separate and multiple analog and digital processors. Additionally, the processor can be implemented using any of a number of architectures. For example, the processor 310 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 350, such as control of user interfaces, applications run by device 350, and wireless communication by device 350.

Processor 352 can communicate with a user through control interface 358 and display interface 356 coupled to a display 354. The display 354 can be, for example, a TFT (Thin-Film-Transistor Liquid Crystal Display) display or an OLED (Organic Light Emitting Diode) display, or other appropriate display technology. The display interface 356 can comprise appropriate circuitry for driving the display 354 to present graphical and other information to a user. The control interface 358 can receive commands from a user and convert them for submission to the processor 352. In addition, an external interface 362 can be provide in communication with processor 352, so as to enable near area communication of device 350 with other devices. External interface 362 can provide, for example, for wired communication in some implementations, or for wireless communication in other implementations, and multiple interfaces can also be used.

The memory 364 stores information within the computing device 350. The memory 364 can be implemented as one or more of a computer-readable medium or media, a volatile memory unit or units, or a non-volatile memory unit or units. Expansion memory 374 can also be provided and connected to device 350 through expansion interface 372, which can include, for example, a SIMM (Single In Line Memory Module) card interface. Such expansion memory 374 can provide extra storage space for device 350, or can also store applications or other information for device 350. Specifically, expansion memory 374 can include instructions to carry out or supplement the processes described above, and can include secure information also. Thus, for example, expansion memory 374 can be provide as a security module for device 350, and can be programmed with instructions that permit secure use of device 350. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 364, expansion memory 374, or memory on processor 352 that can be received, for example, over transceiver 368 or external interface 362.

Device 350 can communicate wirelessly through communication interface 366, which can include digital signal processing circuitry where necessary. Communication interface 366 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 368. In addition, short-range communication can occur, such as using a Bluetooth, Wi-Fi, or other such transceiver (not shown). In addition, GPS (Global Positioning System) receiver module 370 can provide additional navigation- and location-related wireless data to device 350, which can be used as appropriate by applications running on device 350.

Device 350 can also communicate audibly using audio codec 360, which can receive spoken information from a user and convert it to usable digital information. Audio codec 360 can likewise generate audible sound for a user, such as through a speaker, e.g., in a handset of device 350. Such sound can include sound from voice telephone calls, can include recorded sound, e.g., voice messages, music files, etc. and can also include sound generated by applications operating on device 350.

The computing device 350 can be implemented in a number of different forms, as shown in the figure. For example, it can be implemented as a cellular telephone 380. It can also be implemented as part of a smartphone 382, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide, area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

### OTHER EMBODIMENT

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A method for detecting behavioral anomalies in treatment adherence patterns, the method comprising:
obtaining, by one or more computers, one or more first data structures having first fields structuring data that represents (i) an indication that an entity has complied with a therapeutic regimen or (ii) an indication that the entity has not complied with the therapeutic regimen;
determining, by the one or more computers, an initial volatility metric based on the data represented by the one or more first data structures;
determining, by the one or more computers, a central tendency of the initial adherence volatility metric for the entity for at least n-time periods into the future, where n is any non-zero integer;
determining, by the one or more computers, a plurality of boundaries around the central tendency, the plurality of boundaries including a first threshold representing an upper bound of the central tendency and a second threshold representing a lower bound of the central tendency;
obtaining, by the one or more computers, one or more second data structures having second fields structuring data that represents (i) a subsequent indication that an entity has complied with a therapeutic regimen or (ii) a subsequent indication that the entity has not complied with the therapeutic regimen;
determining, by the one or more computers and based on the data represented by the one or more second data structures, a current observed adherence volatility metric;
determining, by the one or more computers, whether the current observed volatility metric satisfies the first threshold or the second threshold; and
based on a determination, by the one or more computers, that the current volatility metric satisfies the first threshold or the second threshold, generating a candidate anomaly data log record, the candidate anomaly data log record including data indicating that a candidate anomaly has been detected.

2. The method of claim 1,
wherein the first fields structuring data that represents (i) an indication that an entity has complied with a therapeutic regimen or (ii) an indication that the entity has not complied with the therapeutic regimen comprises:
data that represents (a) an occurrence of an ingestion of a substance by the entity or (b) an absence of ingestion of a substance by the entity, and
wherein the second fields structuring data that represents (i) a subsequent indication that an entity has complied with a therapeutic regimen or (ii) a subsequent indication that the entity has not complied with the therapeutic regimen comprises:
data that represents (a) a subsequent occurrence of an ingestion of a substance by the entity or (b) a subsequent absence of ingestion of a substance by the entity.

3. The method of claim 2, wherein the one or more first data structures or one or more second data structures were generated, and transmitted, by a mobile device based on ingestion data generated by a patch coupled to the entity.

4. The method of claim 3, wherein the patch generated the ingestion data based on detection, by the patch, of a signal from an ingestible sensor in the substance.

5. The method of claim 4, wherein the substance includes a medicine.

6. The method of claim 1, wherein the upper bound and the lower bound define a region of acceptable adherence volatility metrics.

7. The method of claim 6, wherein determining, by the one or more computers, whether the current observed volatility metric satisfies the first threshold or the second threshold comprises:
continuously obtaining data representing an observed volatility metric; and
comparing the continuously obtained data to the boundaries defined by the first threshold and the second threshold to determine whether the continuously obtained data falls within the region of acceptable adherence volatility metrics.

8. The method of claim 1, wherein determining, by the one or more computers, whether the current observed volatility metric satisfies the first threshold or the second threshold comprises:
evaluating the current observed volatility metric using a binary Markov Chain model to determine whether the current observed volatility metric has exceed the first threshold or the second threshold.

9. The method of claim 1, wherein the adherence volatility metric is based on an entropy rate of Markov parameters.

10. The method of claim 1, wherein the n-time periods into the future includes n-days into the future.

11. The method of claim 1, wherein the n-time periods into the future includes n-hours into the future.

12. A data processing apparatus for method for detecting behavioral anomalies in treatment adherence patterns, comprising:
one or more computers; and
one or more storage devices storing instructions that, when executed by the one or more computers, cause the one or more computers to perform the operations comprising:
obtaining, by the one or more computers, one or more first data structures having first fields structuring data that represents (i) an indication that an entity has complied with a therapeutic regimen or (ii) an indication that the entity has not complied with the therapeutic regimen;
determining, by the one or more computers, an initial volatility metric based on the data represented by the one or more first data structures;
determining, by the one or more computers, a central tendency of the initial adherence volatility metric for the entity for at least n-time periods into the future, where n is any non-zero integer;
determining, by the one or more computers, a plurality of boundaries around the central tendency, the plurality of boundaries including a first threshold representing an upper bound of the central tendency and a second threshold representing a lower bound of the central tendency;
obtaining, by the one or more computers, one or more second data structures having second fields structuring data that represents (i) a subsequent indication that an entity has complied with a therapeutic regimen or (ii) a subsequent indication that the entity has not complied with the therapeutic regimen;
determining, by the one or more computers and based on the data represented by the one or more second data structures, a current observed adherence volatility metric;
determining, by the one or more computers, whether the current observed volatility metric satisfies the first threshold or the second threshold; and
based on a determination, by the one or more computers, that the current volatility metric satisfies the first threshold or the second threshold, generating a candidate anomaly data log record, the candidate anomaly data log record including data indicating that a candidate anomaly has been detected.

13. The system of claim 12,
wherein the first fields structuring data that represents (i) an indication that an entity has complied with a therapeutic regimen or (ii) an indication that the entity has not complied with the therapeutic regimen comprises:
data that represents (a) an occurrence of an ingestion of a substance by the entity or (b) an absence of ingestion of a substance by the entity, and
wherein the second fields structuring data that represents (i) a subsequent indication that an entity has complied with a therapeutic regimen or (ii) a subsequent indication that the entity has not complied with the therapeutic regimen comprises:
data that represents (a) a subsequent occurrence of an ingestion of a substance by the entity or (b) a subsequent absence of ingestion of a substance by the entity.

14. The system of claim 13, wherein the one or more first data structures or one or more second data structures were generated, and transmitted, by a mobile device based on ingestion data generated by a patch coupled to the entity.

15. The system of claim 14, wherein the patch generated the ingestion data based on detection, by the patch, of a signal from an ingestible sensor in the substance.

16. The system of claim 15, wherein the substance includes a medicine.

17. The system of claim 12, wherein the upper bound and the lower bound define a region of acceptable adherence volatility metrics.

18. The system of claim 17, wherein determining, by the one or more computers, whether the current observed volatility metric satisfies the first threshold or the second threshold comprises:
continuously obtaining data representing an observed volatility metric; and
comparing the continuously obtained data to the boundaries defined by the first threshold and the second threshold to determine whether the continuously obtained data falls within the region of acceptable adherence volatility metrics.

19. The system of claim 12, wherein determining, by the one or more computers, whether the current observed volatility metric satisfies the first threshold or the second threshold comprises:
evaluating the current observed volatility metric using a binary Markov Chain model to determine whether the current observed volatility metric has exceed the first threshold or the second threshold.

20. The system of claim 12, wherein the adherence volatility metric is based on an entropy rate of Markov parameters.

21. A non-transitory computer-readable medium storing software comprising instructions executable by one or more computers which, upon such execution, cause the one or more computers to perform the operations comprising:
obtaining, by one or more computers, one or more first data structures having first fields structuring data that represents (i) an indication that an entity has complied with a therapeutic regimen or (ii) an indication that the entity has not complied with the therapeutic regimen;
determining, by the one or more computers, an initial volatility metric based on the data represented by the one or more first data structures;
determining, by the one or more computers, a central tendency of the initial adherence volatility metric for the entity for at least n-time periods into the future, where n is any non-zero integer;
determining, by the one or more computers, a plurality of boundaries around the central tendency, the plurality of boundaries including a first threshold representing an upper bound of the central tendency and a second threshold representing a lower bound of the central tendency;
obtaining, by the one or more computers, one or more second data structures having second fields structuring data that represents (i) a subsequent indication that an entity has complied with a therapeutic regimen or (ii) a subsequent indication that the entity has not complied with the therapeutic regimen;
determining, by the one or more computers and based on the data represented by the one or more second data structures, a current observed adherence volatility metric;
determining, by the one or more computers, whether the current observed volatility metric satisfies the first threshold or the second threshold; and
based on a determination, by the one or more computers, that the current volatility metric satisfies the first threshold or the second threshold, generating a candidate anomaly data log record, the candidate anomaly data log record including data indicating that a candidate anomaly has been detected.

22. The computer-readable medium of claim 21,
wherein the first fields structuring data that represents (i) an indication that an entity has complied with a therapeutic regimen or (ii) an indication that the entity has not complied with the therapeutic regimen comprises:
data that represents (a) an occurrence of an ingestion of a substance by the entity or (b) an absence of ingestion of a substance by the entity, and
wherein the second fields structuring data that represents (i) a subsequent indication that an entity has complied with a therapeutic regimen or (ii) a subsequent indication that the entity has not complied with the therapeutic regimen comprises:
data that represents (a) a subsequent occurrence of an ingestion of a substance by the entity or (b) a subsequent absence of ingestion of a substance by the entity.

23. The computer-readable medium of claim 22, wherein the one or more first data structures or one or more second data structures were generated, and transmitted, by a mobile device based on ingestion data generated by a patch coupled to the entity.

24. The computer-readable medium of claim 23, wherein the patch generated the ingestion data based on detection, by the patch, of a signal from an ingestible sensor in the substance.

25. The computer-readable medium of claim 24, wherein the substance includes a medicine.

26. The computer-readable medium of claim 21, wherein the upper bound and the lower bound define a region of acceptable adherence volatility metrics.

27. The computer-readable medium of claim 26, wherein determining, by the one or more computers, whether the current observed volatility metric satisfies the first threshold or the second threshold comprises:
continuously obtaining data representing an observed volatility metric; and
comparing the continuously obtained data to the boundaries defined by the first threshold and the second threshold to determine whether the continuously obtained data falls within the region of acceptable adherence volatility metrics.

28. The computer-readable medium of claim 21, wherein determining, by the one or more computers, whether the current observed volatility metric satisfies the first threshold or the second threshold comprises:
evaluating the current observed volatility metric using a binary Markov Chain model to determine whether the current observed volatility metric has exceed the first threshold or the second threshold.

29. The computer-readable medium of claim 21, wherein the adherence volatility metric is based on an entropy rate of Markov parameters.
